(19) United States
(12) Patent Application Publication
(10) Pub. No.: US 2021/0228072 A1
(43) Pub. Date: Jul. 29, 2021

(54) TRANSORAL SURGICAL DEVICES AND METHODS

(71) Applicant: Mayo Foundation for Medical Education and Research, Rochester, MN (US)

(72) Inventors: Kathryn M. Van Abel, Rochester, MN (US); Eric J. Moore, Rochester, MN (US)

(73) Assignee: Mayo Foundation for Medical Education and Research, Rochester, MN (US)

(21) Appl. No.: 16/080,450

(22) PCT Filed: Mar. 3, 2017
(86) PCT No.: PCT/US2017/020629

§ 371 Date: Aug. 28, 2018

Related U.S. Application Data
(60) Provisional application No. 62/303,868, filed on Mar. 4, 2016.

(51) Int. Cl. A61B 1/32; A61C 5/90; A61B 17/24; A61B 34/30

(52) U.S. Cl. CPC A61B 1/32; A61C 5/90; A61B 17/1285; A61B 34/30; A61B 17/24

(57) ABSTRACT

Devices and methods can be used for performing transoral surgery. For example, this document provides oral retractor devices and articulating surgical tools that are well-suited for transoral surgery uses. The devices and methods provided herein may be used to treat conditions such as, but not limited to, mouth cancer, throat cancer, tongue cancer, larynx cancer, tonsil cancer, obstructive sleep disorders, and pharyngeal diverticulum, to provide some examples.

### CROSS-REFERENCE TO RELATED APPLICATIONS

This application claims priority to U.S. Application Ser. No. 62/303,868, filed on Mar. 4, 2016. This disclosure of the prior application is considered part of (and is incorporated by reference in) the disclosure of this application.

### BACKGROUND

### 1. Technical Field

This document relates to devices and methods for performing transoral surgery. For example, this document relates to oral retractor devices and articulating surgical tools that are well-suited for transoral surgery uses.

### 2. Background Information

Transoral robotic surgery (TORS) has become one of the leading surgical techniques for management of oropharyngeal malignancy. This technique has also been applied to laryngeal and hypo-pharyngeal pathology, as well sleep medicine surgery. Since the inception of TORS, there has been little advancement in field regarding techniques, exposure, and instrumentation.

TORS is traditionally performed in the supine position with oral retractors that push on the tongue to enable clinicians to view the upper aerodigestive tract through a straight “line-of-sight” approach. However, in some cases this can significantly compress the base of tongue, larynx, and hypopharynx, rendering visualization and making access difficult, and sometimes impractical.

### SUMMARY

This document provides devices and methods for performing transoral surgery. For example, this document provides oral retractor devices and articulating surgical tools that are well-suited for oral surgery uses.

In one implementation, an oral retractor device includes a first maxillary trough configured for engagement with a maxilla of a patient, a first mandibular trough configured for engagement with a mandible of the patient, and a mechanism coupled between the first maxillary trough and the first mandibular trough. The mechanism is actuatable to increase and decrease a separation distance between the first maxillary trough and the first mandibular trough. The first mandibular trough is positionally adjustable in relation to the mechanism and to the first maxillary trough.

Such an oral retractor device may optionally include one or more of the following features. The mechanism may include two arms configured in a scissors arrangement with a pivot joint. The mechanism may include a retention mechanism for releasably latching the two arms in a fixed relative orientation so that the first maxillary trough and the first mandibular trough are separated in a particular fixed separation distance. The mechanism may include an adjust mechanism for positionally adjusting the first mandibular trough in relation to the mechanism and to the first maxillary trough. The oral retraction device may also include a second maxillary trough configured for engagement with the maxilla of a patient and a second mandibular trough configured for engagement with the mandible of the patient. The first maxillary trough and the second maxillary trough may be coupled together by a maxillary arched member. The first mandibular member and the second mandibular member may be coupled together by a mandibular arched member. The mandibular arched member may including an attachment feature for fixating a tongue to the oral retraction device.

In another implementation, an oral retraction system includes an oral retraction device, a maxillary dental protector configured to couple between the maxilla and the first maxillary trough, and a mandibular dental protector configured to couple between the mandible and the first mandibular trough. The oral retraction device may include a first maxillary trough configured for engagement with a maxilla of a patient, a first mandibular trough configured for engagement with a mandible of the patient, and a mechanism coupled between the first maxillary trough and the first mandibular trough. The mechanism is actuatable to increase and decrease a separation distance between the first maxillary trough and the first mandibular trough. The first mandibular trough is positionally adjustable in relation to the mechanism and to the first maxillary trough.

Such an oral retraction system may optionally include one or more of the following features. The maxillary dental protector and the mandibular dental protector may be compliant members configured for protecting teeth. The mechanism may include two arms configured in a scissors arrangement with a pivot joint. The mechanism may include a retention mechanism for releasably latching the two arms in a fixed relative orientation so that the first maxillary trough and the first mandibular trough are separated in a particular fixed separation distance. The mechanism may include an adjust mechanism for positionally adjusting the first mandibular trough in relation to the mechanism and to the first maxillary trough.

In another implementation, a method of retracting a mouth of a patient in an open position includes: (a) engaging a first maxillary trough of an oral retraction device with a maxilla of the patient; (b) engaging a first mandibular trough of the oral retraction device with a mandible of the patient; (c) actuating a separation mechanism of the oral retraction device to increase a separation distance between the first maxillary trough and the first mandibular trough; and (d) actuating a mandibular trough adjustment mechanism to move the mandible in an anterior direction in relation to the maxilla.

Such a method of retracting a mouth of a patient in an open position may optionally include one or more of the following features. The actuating the separation mechanism may include releasably latching the first maxillary trough and the first mandibular trough in a particular fixed separation distance. The method may also include: (e) placing a maxillary dental protector between the maxilla and the first maxillary trough; and (f) placing a mandibular dental protector between the mandible and the first mandibular trough.

Particular embodiments of the subject matter described in this document can be implemented to realize one or more of the following advantages. In some embodiments, the devices and methods provided herein provide better transoral access to target operative areas as compared to conventional devices and techniques. The devices and methods can provide better transoral access, for example, by moving the mandible forward in relation to the maxilla. In doing so, the patient's tongue also moves forward, providing better access. In some embodiments, various mouth and throat conditions can be treated in a minimally invasive fashion using the devices and methods provided herein. Such minimally invasive techniques can reduce recovery times, patient discomfort, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a perspective view of an example oral retractor device in accordance with some embodiments.

FIG. 2 is a perspective view of a retractor tool that can be used in conjunction with the oral retractor device of FIG. 1.

FIG. 3 shows an example clip application device in accordance with some embodiments.

FIG. 4 shows an example grasping device in accordance with some embodiments.

FIG. 5 shows an example bipolar cautery device in accordance with some embodiments.

FIG. 6 is an alternative distal end of the bipolar cautery device of FIG. 5.

FIG. 7 is another alternative distal end of the bipolar cautery device of FIG. 5.

FIG. 8 is an example monopolar cautery device in accordance with some embodiments.

Like reference numbers represent corresponding parts throughout.

### DETAILED DESCRIPTION

This document provides devices and methods for performing transoral surgery. For example, this document provides oral retractor devices and articulating surgical tools that are well-suited for oral surgery uses. The devices and methods provided herein may be used to treat conditions such as, but not limited to, mouth cancer, throat cancer, tongue cancer, larynx cancer, tonsil cancer, obstructive sleep disorders, and pharyngeal diverticulum, to provide some examples.

In some cases, the devices and methods provided herein can be used for transoral robotic surgery (TORS). TORS is a procedure to remove oral cancers, for example, in which a surgeon uses a sophisticated, computer-enhanced system to guide the surgical tools. TORS gives the surgeon an enhanced view of the cancer and surrounding tissue. Using a robotic system to guide the surgical tools allows for more-precise movements in tiny spaces and the capability to work around corners. When compared with more-traditional procedures, TORS for oropharyngeal, laryngeal and hypo-pharyngeal cancer can tend to result in a quicker recovery and fewer complications.

In some cases, the devices and methods provided herein can be used for seated TORS procedures. That is, in some cases rather than performing TORS on a patient in the supine position, TORS can be performed on the patient can be in a seated position. In addition, transoral surgery (TOS) in the seated position can also be accomplished with endoscopes and hand held instrumentation, without robotic assistance. The devices and methods provided herein can be used each of these techniques.

This disclosure provides devices and methods such as a new oral retractor device that can expose the operative field without compression of the patient's tongue. New oral retracting instruments that can be hand held, fixed, or used with the robotic arm (for TORS) to assist retracting the tongue in a more surgically ergonomic position as also provided. The oral retractor devices provided herein pulls the tongue forward. In addition, new instruments that can be used for surgical access (e.g., in the seated position and other positions) such as angled/adjustable clip appliers, angled/adjustable bipolar cautery, angled/adjustable dissectors, and the like, are also provided herein. The instruments are configured to curve in correspondence with the curve at the back of the mouth and pharynx and adjust to accommodate individual patient and tumor anatomy.

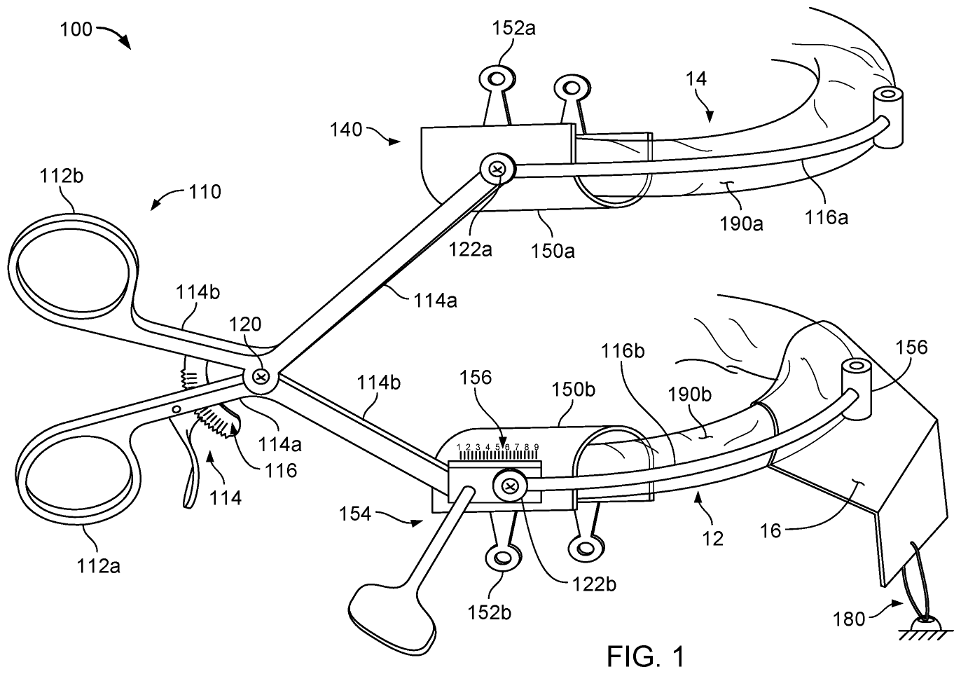

Referring to FIG. 1, an example oral retractor device 100 can be used to retract the mouth of a patient in an open position in preparation for transoral surgery. Oral retractor device 100 is configured to provide advantageous exposure and visualization of the complex anatomy of the back of the mouth and of the upper aerodigestive tract. That is accomplished, for example, because oral retractor device 100 allows the patient's mandible 12 to be distracted in a cranial-caudal (open/close) direction, as well as in a protracted anterior-posterior (jaw forward/back) position in relation to the patient's maxilla 14.

In some embodiments, the tongue 16 can be pulled anteriorly by a tongue stitch 180. In some cases, tongue stitch 180 can be intermittently secured to an attachment member 156 on the anterior/inferior arch of oral retractor device 100 to maximize exposure without locking it into any single position. This would allow both the surgeon and surgical assistant to manipulate tongue 16 to gain better visibility and access throughout the case.

In the depicted embodiment, oral retractor device 100 includes an actuation portion 110 and a retraction portion 140. Actuation portion 110 and retraction portion 140 are coupled at a pivot joint 120. Hence, actuation portion 110 and retraction portion 140 expand and retract inversely to each other.

Actuation portion 110 includes a first handle 112a and a second handle 112b. First handle 112a is connected to a first arm 114a. Second handle 112b is connected to a second arm 114b. Arms 114a and 114b cross at a pivot joint 120 (like a scissors). Therefore, as actuation portion 110 is compressed to a smaller configuration (i.e., as handles 112a and 112b are moved closer to each other), retraction portion 140 becomes correspondingly enlarged. Conversely, as actuation portion 110 is enlarged (i.e., as handles 112a and 112b are moved farther from each other), retraction portion 140 transforms to a correspondingly smaller configuration. Therefore, to retract (separate) mandible 12 in an open position in relation to maxilla 14, a clinician can compress handles 112a and 112b towards each other.

Actuation portion 110 also includes a retention mechanism 114. Retention mechanism 114 is releasably latchable. That is, as handles 112a and 112b are compressed towards each other, retention mechanism 114 can be biased to releasably latch first handle 112a in a fixed position in relation to second handle 112b. Accordingly, the retraction forces applied by retraction portion 140 to mandible 12 and maxilla 14 are maintained. In that fashion, the patient's mouth can be maintained retracted in an open position. The retraction forces can also be selectively released by deactivating (e.g., unlatching) retention mechanism 114.

In some embodiments, retention mechanism 114 also includes a numeric scale 116. Numeric scale 116 can provide a quantitative indication of the extent of retraction between mandible 12 and maxilla 14. In some embodiments, a force sensor is included that provides the clinician with an indication of the retraction force applied by retraction portion 140 onto the patient's mandible 12 and maxilla 14.

Retraction portion 140 includes arms 114a and 114b. Arms 114a and 114b extend from pivot joint 120 to a maxillary joint 122a and a mandibular joint 122b. In some embodiments, maxillary joint 122a and/or mandibular joint 122b are pivoting joints or hinged junctions. In some embodiments, a maxillary arch member 116a extends from maxillary joint 122a, and/or a mandibular arch member 116b extends from mandibular joint 122b.

A maxillary trough 150a is pivotably coupled to maxillary joint 122a. A mandibular trough 150b is pivotably coupled to mandibular joint 112b. Troughs 150a and 150b releasably couple with the patient's teeth or jaw bones. For example, in some cases troughs 150a and 150b releasably couple with the maxillary and mandible molars. In some embodiments, troughs 150a and 150b can be relocated to other positions on arch member 116a and 116b respectively.

In some cases, a maxillary dental protector 190a and a mandibular dental protector 190b are used between troughs 150a and 150b and the patient's maxillary and mandibular dentition respectively. Dental protectors 190a and 190b protect the teeth and tongue 16, and can prevent or inhibit slipping of the retractor troughs 150a and 150b in relation to the teeth in some embodiments.

In some embodiments, dental protectors 190a and 190b can be compliant members similar to mouth guards (i.e., worn on the teeth). Mandibular trough 150b releasably couples with mandibular dental protector 190b (such that mandibular distraction can be actuated by mandibular trough 150b, as described further below). In some cases, dental protectors 190a and 190b are inserts that are attachable to troughs 150a and 150b. Such inserts can be disposable items that can be removed from attachment to troughs 150a and 150b after use and discarded (while oral retractor device 100 can be reused after decontamination).

Troughs 150a and 150b apply the retraction force to separate maxilla 14 and mandible 12. In some embodiments, a single maxillary trough 150a and a single mandibular trough 150b are included as part of oral retractor device 100. In some such embodiments, no maxillary arch member 116a or mandibular arch member 116b are included as part of oral retractor device 100. When a single maxillary trough 150a and a single mandibular trough 150b are included as part of oral retractor device 100, either a single oral retractor device 100 can be used for a patient, or two oral retractor devices 100 can be used for a patient (one on each side of the patient's mouth). In such a case, the second oral retractor device 100 can be a mirror image of the oral retractor device 100 as shown. In such a case, no maxillary arch member 116a and mandibular arch member 116b are included as part of oral retractor device 100.

Alternatively, in some embodiments two maxillary troughs 150a and two mandibular troughs 150b are included as part of a bilateral oral retractor device 100. In some such embodiments, maxillary arch member 116a extends between the two maxillary troughs 150a, and mandibular arch member 116b extends between the two mandibular troughs 150b. Accordingly, each of both maxillary troughs 150a and each of both mandibular troughs 150b can apply retraction forces to open the patient's mouth.

In some embodiments, maxillary arch member 116a and/or mandibular arch member 116b are adjustable to allow for a customized fit for a particular patient's mouth (e.g., the ability to adjust to different mandibular/maxillary arch widths). For example, maxillary arch member 116a and/or mandibular arch member 116b can include one or more hinges, telescopic joints, and the like. In some embodiments, maxillary arch member 116a and/or mandibular arch member 116b can be malleable members that can be custom formed to provide a suitable fit for a particular patient's mouth size and shape.

In some embodiments, mandibular arch member 116b includes an attachment member 156 that can be used to secure tongue 16 in an extended orientation, so that tongue 16 is out of the way for optimal access to the throat. For example, in some cases a suture stitch can be used to attach tongue 16 to attachment member 156.

In some embodiments, maxillary trough 150a and/or mandibular trough 150b include one or more through-holes 152a and 152b respectively. Through-holes 152a and 152b can be used, along with bone screws, to rigidly couple maxillary trough 150a to maxilla 14 and/or mandibular trough 150b to mandible 12 respectively. In some cases, the use of self-tapping screws in conjunction with through-holes 152a and 152b may be advantageous for stabilization of oral retraction device 100 in a dentulous or edentulous patient, and would result in limited instrumentation/crowding within the oral cavity.

In some embodiments, retraction portion 140 includes a mandibular trough adjustment mechanism 154. Using mandibular trough adjustment mechanism 154, mandibular trough 150b can be positionally adjusted in relation to maxillary trough 150a to increase or decrease mandibular distraction. That is, mandible 12 can be moved anteriorly and posteriorly in relation to maxilla 14 by adjusting mandibular trough adjustment mechanism 154. By moving mandible 12 anteriorly in relation to maxilla 14, access to the rear of the mouth and throat is generally enhanced.

Mandibular trough adjustment mechanism 154 can be a variety of different types of mechanisms. For example, in some embodiments mandibular trough adjustment mechanism 154 can be a rack and pinion gear arrangement. In some embodiments, mandibular trough adjustment mechanism 154 can be a lead screw arrangement. In some embodiments, mandibular trough adjustment mechanism 154 includes a ratcheting mechanism.

In some embodiments, mandibular trough adjustment mechanism 154 facilitates an adjustment up to about 1 cm of travel. In some embodiments, mandibular trough adjustment mechanism 154 facilitates an adjustment up to about 2 cm of travel.

Mandibular trough adjustment mechanism 154 can include a numeric mandible distraction scale 156. Mandible distraction scale 156 can provide a quantitative indication of the extent of the mandibular distraction. In some embodiments, a force sensor is included that provides the clinician with an indication of the distraction force applied by mandibular trough adjustment mechanism 154 onto the patient's mandible 12.

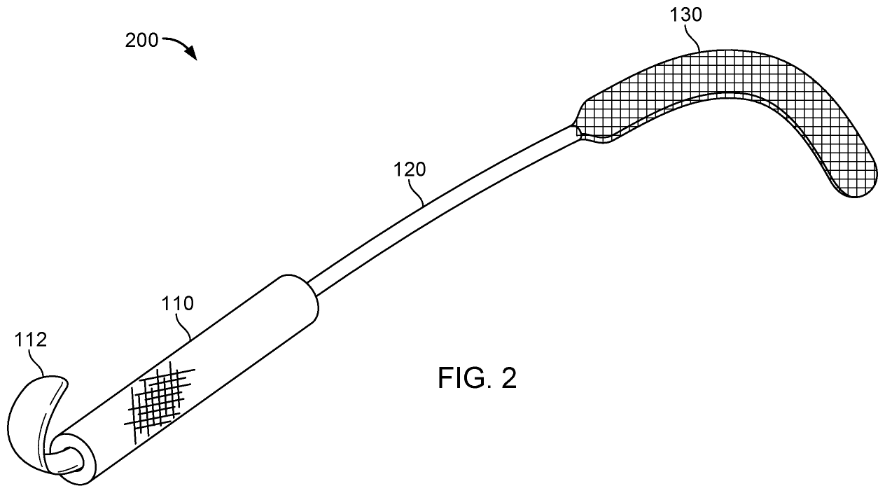

Referring also to FIG. 2, a transoral retractor instrument 200 can be used to apply additional retraction to tongue 16. The transoral retractor may have one retractor blade (as pictured) or in some embodiments may have two. These may be adjustable or fixed. In some embodiments, transoral retractor instrument 200 is used by attaching it to oral retraction device 100. In some embodiments, transoral retractor instrument 200 is used manually by a clinician as a separate instrument.

In the depicted embodiment, transoral retractor instrument 200 includes a handle 110, a shaft 120, and an end effector 130. Shaft 120 extends between handle 110 and end effector 130.

Handle 110 can be configured for manual gripping. For example, handle 110 can have a non-slip surface (e.g., rubberized, contoured, or textured). In addition, in some embodiments a hook 112 can extend proximally from handle 110. Hook 112 can provide a rigid lateral surface to which a clinician's hand can abut.

End effector 130 can be configured to apply retraction to tongue 16, especially at the base of tongue 16. Accordingly, end effector 130 can be curved, and can be textured to increase the friction between end effector 130 and tongue 16. End effector 130 can have rounded edges to provide atraumatic retraction.

End effector 130 can be made available in various sizes and shapes. That is, end effector 130 can be made in various lengths, widths, and angles. In some cases, a set of transoral retractor instrument 200 in various sizes and/or configurations can be made available. In some embodiments, retractor instrument 200 is adjustable.

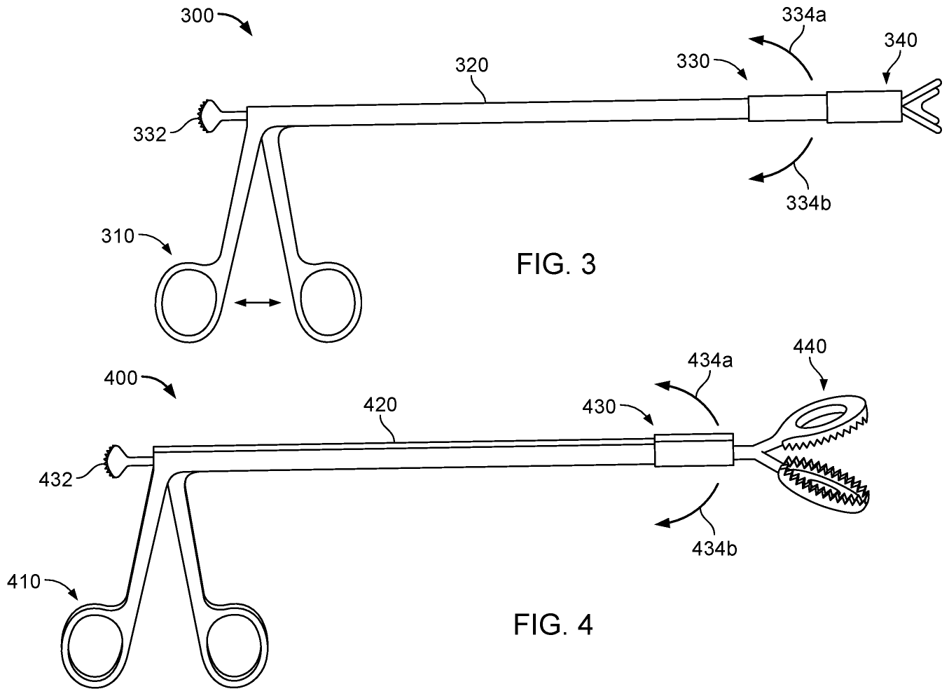

Referring also to FIG. 3, in some cases an example multi-fire clip applier 300 can be advantageously used in conjunction with oral retraction device 100. Multi-fire clip applier 300 can apply clips to attenuate bleeding, for example.

Multi-fire clip applier 300 includes a handle 310, a shaft 320, an articulating portion 330, and an end effector 340. Handle 310 can be used to actuate end effector 340 (i.e., to apply clips).

Articulating portion 330 can be used to position end effector 340 in various positions relative to shaft 320. In some embodiments, articulating portion 330 can swing/pivot end effector 340 through an arc of about 180 degrees (as indicated by arrows 334a and 334b). This articulation functionality can be advantageous for working in or past the curved space the at the back of the mouth, pharynx (near the base of tongue 16), and larynx. The articulation of articulating portion 330 can be adjusted by a clinician using an articulation adjustment mechanism 332.

Referring also to FIG. 4, in some cases an example grasper 400 can be advantageously used in conjunction with oral retraction device 100. Grasper 400 can be used to retract tissue in the throat area, for example.

Grasper 400 includes a handle 410, a shaft 420, an articulating portion 430, and an end effector 440. Handle 410 can be used to actuate end effector 440 (i.e., to open and close the jaws).

Articulating portion 430 can be used to position end effector 440 in various positions relative to shaft 420. In some embodiments, articulating portion 430 can swing/pivot end effector 440 through an arc of about 180 degrees (as indicated by arrows 434a and 434b). This articulation functionality can be advantageous for working in or past the curved space the at the back of the mouth and pharynx (near the base of tongue 16). The articulation of articulating portion 430 can be adjusted by a clinician using an articulation adjustment mechanism 432.

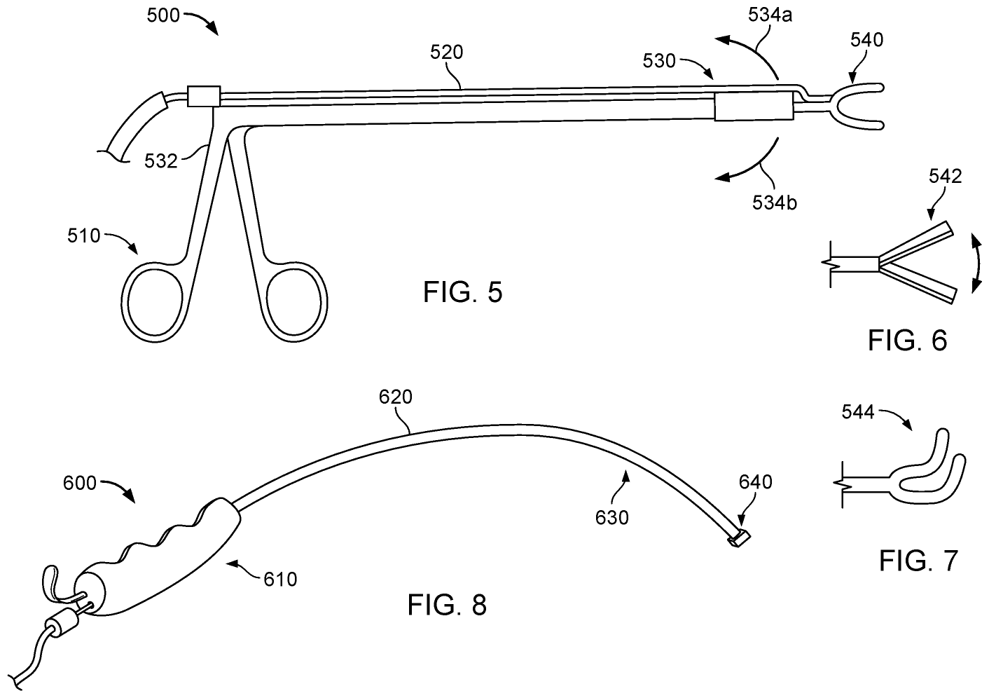

Referring also to FIG. 5, in some cases an example bipolar cautery device 500 can be advantageously used in conjunction with oral retraction device 100. Bipolar cautery device 500 can be used to cauterize tissue (e.g., blood vessels) in the throat area, for example. In some embodiments, the cauterization can be activated using a foot pedal (not shown).

Bipolar cautery device 500 includes a handle 510, a shaft 520, an articulating portion 530, and an end effector 540. In some embodiments, handle 510 can be used to adjust end effector 540. In some embodiments, handle 510 can be used to adjust the position of articulating portion 530. In the depicted embodiment, end effector 540 is a static straight bipolar cauterization member.

Articulating portion 530 can be used to position end effector 540 in various positions relative to shaft 520. In some embodiments, articulating portion 530 can swing/pivot end effector 540 through an arc of about 180 degrees (as indicated by arrows 534a and 534b). This articulation functionality can be advantageous for working in or past the curved space the at the back of the mouth and pharynx (near the base of tongue 16). In some embodiments, the articulation of articulating portion 530 can be adjusted by a clinician using an articulation adjustment mechanism 532.

FIGS. 6 and 7 provide alternative bipolar cauterization members that can be used for bipolar cautery device 500 (instead of end effector 540). End effector 542 is a dynamic straight bipolar cauterization member that can be actuated open and closed. End effector 544 is a static curved bipolar cauterization member.

Referring also to FIG. 8, in some cases an example monopolar cautery device 600 can be advantageously used in conjunction with oral retraction device 100. Monopolar cautery device 600 can be used to cauterize tissue (e.g., blood vessels) in the throat area, for example. In some embodiments, the cauterization can be activated using a foot pedal (not shown).

Monopolar cautery device 600 includes a handle 610 (e.g., pistol grip), a curved shaft 620, an optional articulating portion 630, and an end effector 640.

Optional articulating portion 630 can be used to position end effector 640 in various positions relative to shaft 620. In some embodiments, articulating portion 630 can swing/pivot end effector 640 through an arc of about 180 degrees. This optional articulation functionality can be advantageous for working in or past the curved space the at the back of the mouth, pharynx (near the base of tongue 16), and larynx.

Shaft 620 can be curved to allow access around other instruments. In some embodiments, shaft 620 is laterally rigid to allow some pressure to be applied by shaft 620 to tissues. In some embodiments, the distal end portion of shaft 620 can have a bend radius that is less than the proximal end portion of shaft 620. This bend in the distal end portion of shaft 620 can be advantageous for working in or past the curved space the at the back of the mouth, pharynx (near the base of tongue 16), and larynx. In some cases, various monopolar cautery devices 600 with different configurations of shaft 620 (e.g., different lengths, bends, etc.) can be available.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products. Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. An oral retraction device comprising:
a first maxillary trough configured for engagement with a maxilla of a patient;
a first mandibular trough configured for engagement with a mandible of the patient; and
a mechanism coupled between the first maxillary trough and the first mandibular trough, wherein the mechanism is actuatable to increase and decrease a separation distance between the first maxillary trough and the first mandibular trough, and wherein the first mandibular trough is positionally adjustable in relation to the mechanism and to the first maxillary trough.

2. The oral retraction device of claim 1, wherein the mechanism includes two arms configured in a scissors arrangement with a pivot joint.

3. The oral retraction device of claim 2, wherein the mechanism includes a retention mechanism for releasably latching the two arms in a fixed relative orientation so that the first maxillary trough and the first mandibular trough are separated in a particular fixed separation distance.

4. The oral retraction device of claim 3, wherein the mechanism includes an adjust mechanism for positionally adjusting the first mandibular trough in relation to the mechanism and to the first maxillary trough.

5. The oral retraction device of claim 1, further comprising a second maxillary trough configured for engagement with the maxilla of a patient and a second mandibular trough configured for engagement with the mandible of the patient.

6. The oral retraction device of claim 5, wherein the first maxillary trough and the second maxillary trough are coupled together by a maxillary arched member, and wherein the first mandibular member and the second mandibular member are coupled together by a mandibular arched member.

7. The oral retraction device of claim 6, wherein the mandibular arched member includes an attachment feature for fixating a tongue to the oral retraction device.

8. An oral retraction system comprising:
an oral retraction device comprising:
a first maxillary trough configured for engagement with a maxilla of a patient;
a first mandibular trough configured for engagement with a mandible of the patient; and
a mechanism coupled between the first maxillary trough and the first mandibular trough, wherein the mechanism is actuatable to increase and decrease a separation distance between the first maxillary trough and the first mandibular trough, and wherein the first mandibular trough is positionally adjustable in relation to the mechanism and to the first maxillary trough;
a maxillary dental protector configured to couple between the maxilla and the first maxillary trough; and
a mandibular dental protector configured to couple between the mandible and the first mandibular trough.

9. The oral retraction system of claim 8, wherein the maxillary dental protector and the mandibular dental protector are compliant members configured for protecting teeth.

10. The oral retraction system of claim 8, wherein the mechanism includes two arms configured in a scissors arrangement with a pivot joint.

11. The oral retraction system of claim 10, wherein the mechanism includes a retention mechanism for releasably latching the two arms in a fixed relative orientation so that the first maxillary trough and the first mandibular trough are separated in a particular fixed separation distance.

12. The oral retraction system of claim 11, wherein the mechanism includes an adjust mechanism for positionally adjusting the first mandibular trough in relation to the mechanism and to the first maxillary trough.

13. A method of retracting a mouth of a patient in an open position, the method comprising:
engaging a first maxillary trough of an oral retraction device with a maxilla of the patient;
engaging a first mandibular trough of the oral retraction device with a mandible of the patient;
actuating a separation mechanism of the oral retraction device to increase a separation distance between the first maxillary trough and the first mandibular trough; and
actuating a mandibular trough adjustment mechanism to move the mandible in an anterior direction in relation to the maxilla.

14. The method of claim 13, wherein the actuating the separation mechanism includes releasably latching the first maxillary trough and the first mandibular trough in a particular fixed separation distance.

15. The method of claim 13, further comprising:
placing a maxillary dental protector between the maxilla and the first maxillary trough; and
placing a mandibular dental protector between the mandible and the first mandibular trough.
